(19) United States
(12) Patent Application Publication
(10) Pub. No.: US 2018/0311204 A1
(43) Pub. Date: Nov. 1, 2018

(54) METHOD FOR PREVENTING OR TREATING HEART FAILURE

(71) Applicant: GUANGZHOU MEDICAL UNIVERSITY, Guangzhou (CN)

(72) Inventor: Xiaoqian Wu, Guangzhou (CN)

(21) Appl. No.: 15/965,860

(22) Filed: Apr. 28, 2018

(30) Foreign Application Priority Data
Apr. 28, 2017 (CN) 201710299155.3

(51) Int. Cl. A61K 31/352; A61P 9/04

(52) U.S. Cl. CPC A61K 31/352; A61P 9/04

(57) ABSTRACT

The present invention discloses a method for preventing or treating heart failure, relating to a novel use of nobiletin, the nobiletin reverses cardiac remodeling after heart failure and ischemia, the reversion of cardiac remodeling caused by ischemic heart failure includes reversing the increase of systolic ventricular internal dimension, reversing the decrease of ventricular ejection fraction and ventricular fractional shortening, reducing infarct size, and promoting cardiomyocyte survival rate. The present invention reveals the use of nobiletin in preventing or treating heart failure.

### CROSS-REFERENCE TO RELATED APPLICATIONS

The present application claims the benefit of Chinese Patent Application No. 201710299155.3 filed on Apr. 28, 2017, the contents of which are hereby incorporated by reference.

### FIELD OF THE INVENTION

The present invention relates to a method for preventing or treating heart failure, specifically, by administrating nobiletin to a patient.

### BACKGROUND OF THE INVENTION

Chronic heart failure is a pathophysiological condition when the heart is unable to pump sufficient blood to meet the body's need of metabolism due to heart dysfunction caused by cardiac overload, limited diastole, virus infection, anemia and so on. Chronic heart failure presents with cardiac remodeling which is considered as a fundamental mechanism resulting in heart failure.

Nobiletin, derived from Citrus nobilis Lour., is isolated by processes like ethanol extraction, chromatography, crystallization, etc. Nobiletin has been found to have several pharmacological activities including anti-hemagglutination, anti-thrombus, anti-carcinoma, anti-fungus, anti-inflammation, anti-allergy, anti-cholinesterase, anti-epilepsy, and also act as an accelerant to carbohydrate metabolism.

In present, there are various of medications for chronic heart failure, for example, diuretics, angiotensin coverting enzyme inhibitors, digoxin, etc.; however, efficacy of nobiletin in treating chronic heart failure has not yet been reported.

### SUMMARY OF THE INVENTION

The present invention provides a use of nobiletin for preventing or treating heart failure which overcomes drawbacks of the prior art in the field. The technical scheme of the present invention relates to a method for preventing or treating heart failure comprising administrating nobiletin to a patient.

Preferably, the nobiletin prevents or reverses cardiac remodeling resulting from chronic heart failure and ischemia.

More preferably, the reversion of cardiac remodeling caused by heart failure and ischemia includes reversing increased systolic ventricular internal dimensions, reversing decreased ventricular ejection fractions and ventricular fractional shortenings, reducing infarct sizes and promoting survival rates of cardiomyocytes.

Most preferably, the heart failure is chronic heart failure.

In addition, the present invention provides a pharmaceutical composition comprising nobiletin and pharmaceutically acceptable carriers.

Preferably, the pharmaceutical is in form of an injection or a lyophilized product.

In aforementioned embodiments, the pharmaceutical composition can be in various forms including liquid, semisolid and solid, for example, liquid solutions (e.g., injections and infusions), dispersants or suspensions, tablets, pills, powders, liposomes and suppositories. Preferable forms depend on predetermined drug delivery routes and therapeutic application. A typical form of the pharmaceutical composition is an injection or an infusion. A preferable route of drug delivery is a parenteral (e.g., intravenous, subcutaneous, intraperitoneal, intramuscular) administration. In a preferable embodiment, the pharmaceutical composition is administrated by intraperitoneal injection. Auxiliary active compounds can also be added to the pharmaceutical composition.

In aforementioned embodiments, the pharmaceutically acceptable carriers include any and all biocompatible solvents, dispensing medium, coatings, antibacterial and antifungal agents, isotonic agents, delayed release agents and so on. Examples of pharmaceutically acceptable carriers encompass any of water, normal saline, phosphate buffer, dextran, glycerine, ethanol or a combination thereof. In many occasions, a preferable pharmaceutical composition contains an isotonic agent such as saccharide, polyhydric alcohols or sodium chloride. Pharmaceutically acceptable carriers can also include a handful of adjuvants like humectants or emulsifier, preservatives or buffer which can extend the shelf life or efficacy of antibodies or antibody fragments.

Besides, the present invention also provides a method for preventing or treating heart failure comprising administrating the pharmaceutical composition to a patient.

Preferably, the pharmaceutical composition prevents or reverses cardiac remodeling resulting from chronic heart failure and ischemia.

More preferably, the reversion of cardiac remodeling caused by heart failure and ischemia includes reversing increased systolic ventricular internal dimensions, reversing decreased ventricular ejection fractions and ventricular fractional shortenings, reducing infarct sizes and promoting survival rates of cardiomyocytes.

Most preferably, the heart failure is chronic heart failure.

The present invention provides a method of nobiletin in preventing or treating heart failure, by the use of laboratory model of myocardial ischemia and experiments on cardiomyocytes subjected to oxygen-glucose deprivation, the present invention demonstrates that nobiletin is capable of reversing cardiac remodeling caused by myocardial ischemia including reversing the increased of systolic ventricular internal dimensions, reversing the decreased of ventricular ejection fractions and ventricular fractional shortenings, reducing infarct sizes and promoting survival rates of cardiomyocytes, that nobiletin has therapeutic effect on chronic heart failure and can be used as a drug for chronic heart failure

### BRIEF DESCRIPTION OF DRAWINGS

FIG. 1 shows a left anterior descending coronary artery of a rat in Experiment 1 before ligation.

FIG. 2 shows that ST segments rose in 2-lead electrocardiogram of a rat after ligation of the left anterior descending coronary artery.

FIG. 3 shows that distal myocardial tissue turned pale after the ligation of the left anterior descending coronary artery of a rat in Experiment 1.

FIG. 4 shows the echocardiograms of Experiment 1.

FIG. 5A shows a histogram of left ventricular ejection fractions; FIG. 5B shows a histogram of left ventricular fractional shortenings; wherein NS represents P>0.05, * represents P<0.05, *** represents P<0.001.

FIG. 6A shows a histogram of left ventricular internal dimensions, systolic;

FIG. 6B shows a histogram of left ventricular internal dimensions, diastolic; wherein NS represents P>0.05, * represents P<0.05, ** represents P<0.01, *** represents P<0.001.

FIG. 7 shows images of TTC staining of rat myocardia in Experiment 2.

FIG. 8 shows a histogram of infarction area fractions of rats in Experiment 2, wherein ** represents P<0.01, *** represents P<0.001.

FIG. 9A shows a scatter plot of cardiomyocytes subjected to oxygen-glucose deprivation (OGD) (as control).

FIG. 9B shows a scatter plot of OGD-subjected cardiomyocytes added with 5 μM nobiletin (NOB-5 μM).

FIG. 9C shows a scatter plot of OGD-subjected cardiomyocytes added with 10 μM nobiletin (NOB-10 μM).

FIG. 9D shows a scatter plot of OGD-subjected cardiomyocytes added with 20 μM nobiletin (NOB-20 μM).

FIG. 9E shows a scatter plot of OGD-subjected cardiomyocytes added with 40 μM nobiletin (NOB-40 μM).

FIG. 9F shows a scatter plot of OGD-subjected cardiomyocytes added with 80 μM nobiletin (NOB-80 μM).

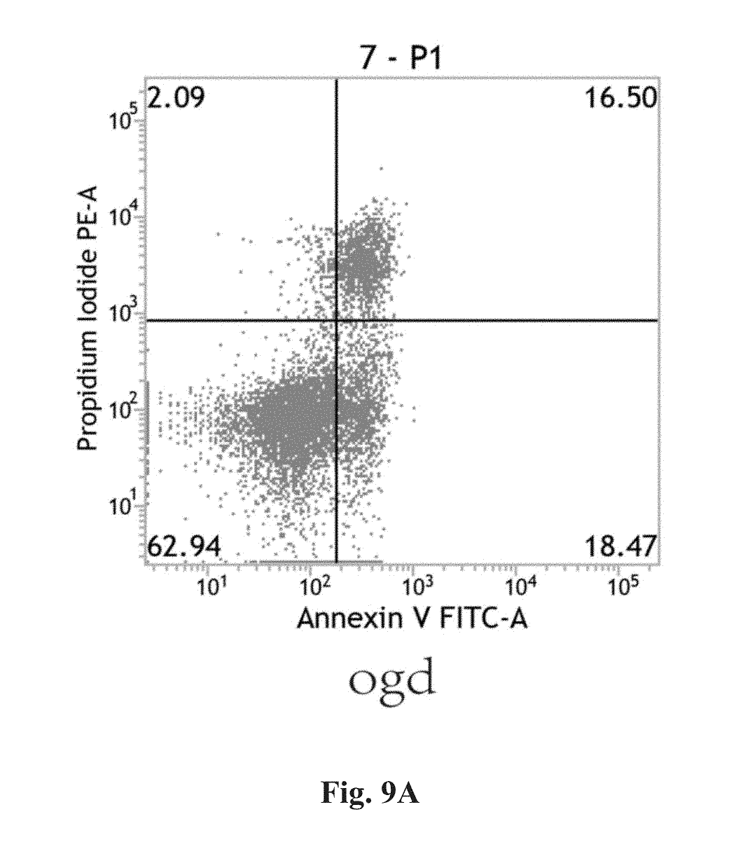

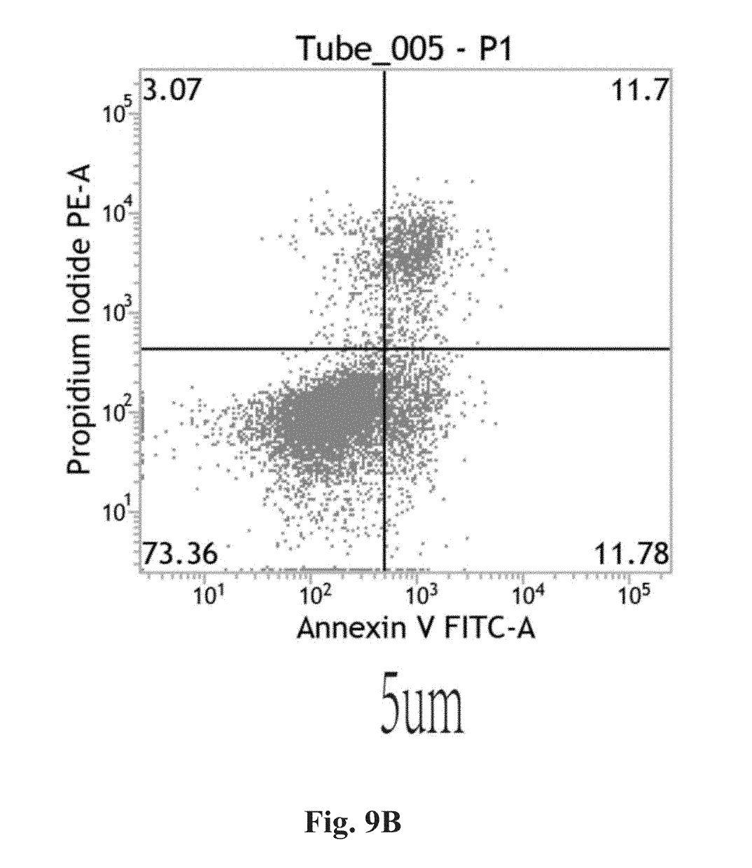

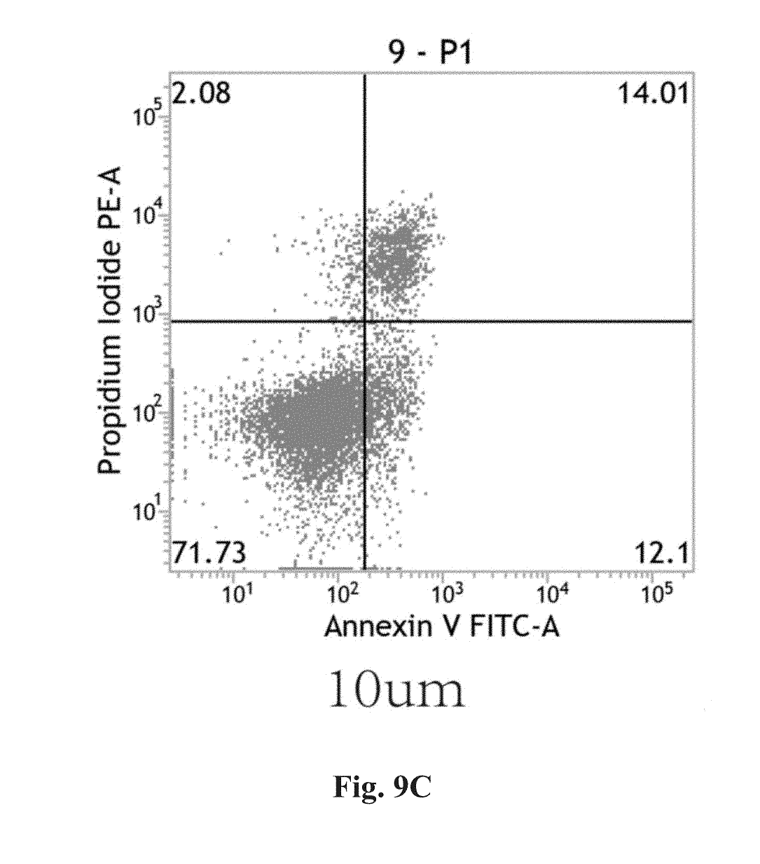

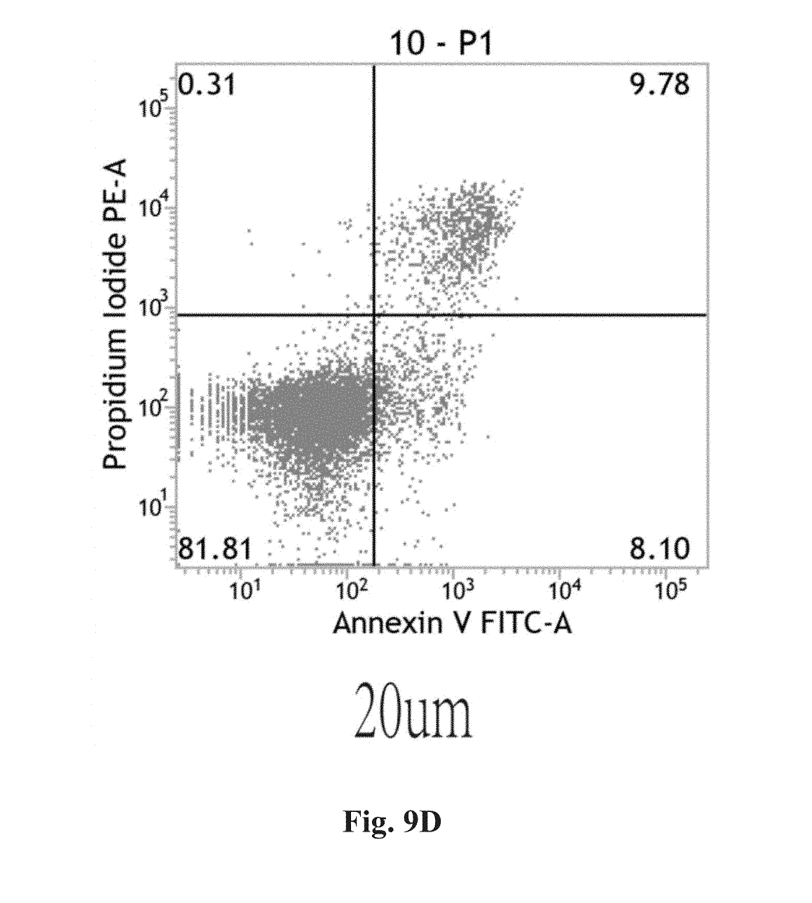

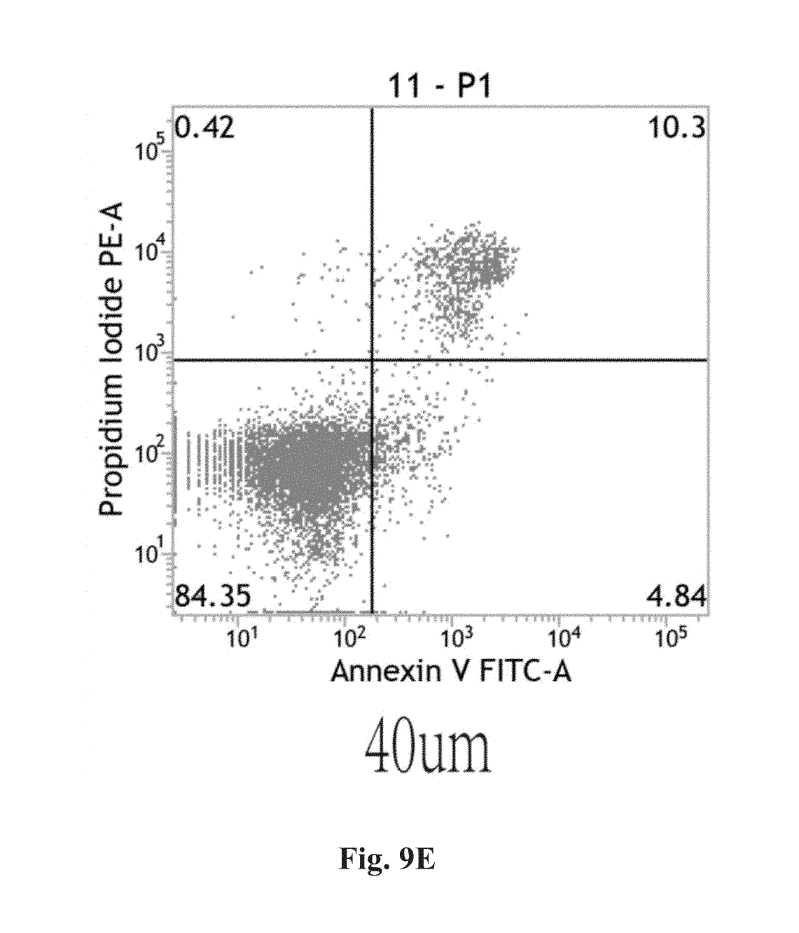

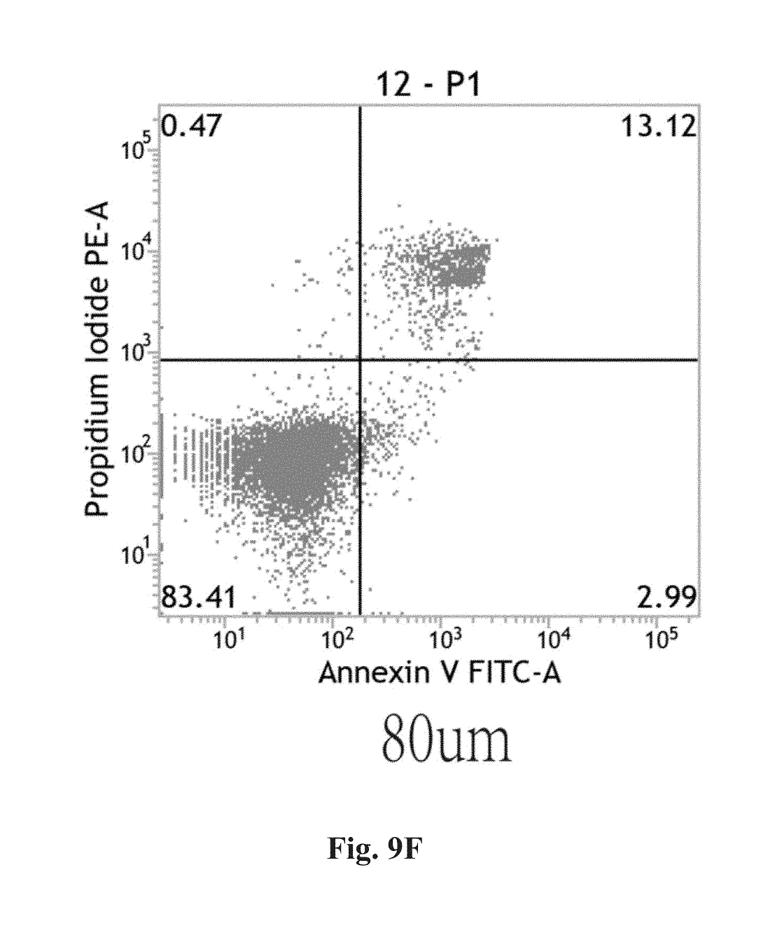

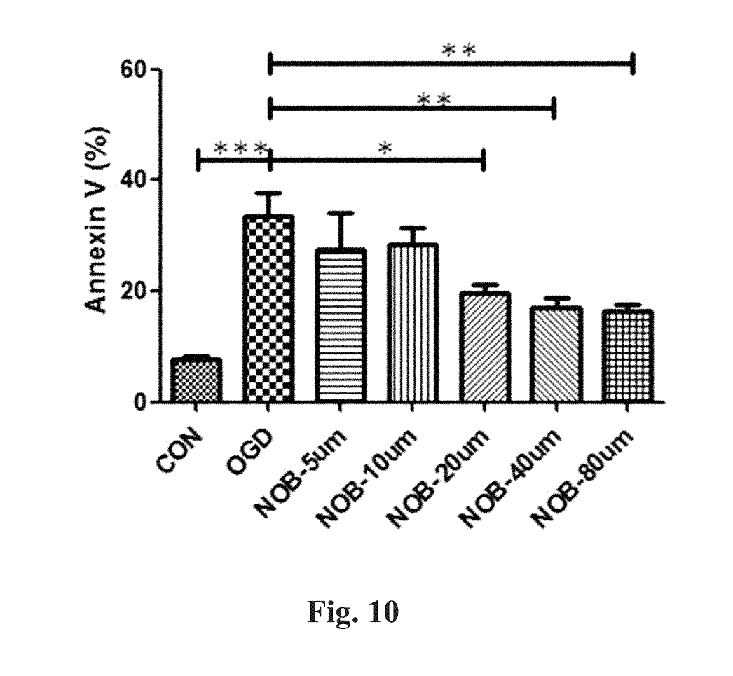

In FIGS. 9A-9F, green fluorescence intensity of AnnexinV was plotted by on horizontal axis and red fluorescence intensity of propidium iodide (PI) on the vertical. FIG. 10 shows statistic results of OGD-subjected cardiomyocytes of Experiment 3, wherein * represents P<0.05, ** represents P<0.01, *** represents P<0.001.

### DETAILED DESCRIPTION OF ILLUSTRATED EMBODIMENTS

In order to better reveal the purpose, technique scheme and advantages of the present invention, the present invention will be further illustrated in conjunction with specific embodiments, tables and drawings

Experiment 1: Effect of Nobiletin on Ventricle.

60 rats were randomized into 6 groups: (i) sham group (Sham), n=10, (left anterior descending (LAD) coronary arteries of the rats were only laid upon sutures without ligation, and 0.5% Tween normal saline was injected intraperitoneally); (ii) infarction group (AMI), n=10, (LAD coronary arteries of the rats were ligated before 0.5% Tween normal saline was injected intraperitoneally); (iii) nobiletin group of low dosage (AMI+NOB-L, n=10; LAD coronary arteries of the rats were ligated before 7.5 mg/kg nobiletin was injected intraperitoneally); (v) nobiletin group of medium dosage (AMI+NOB-M), n=10, (LAD coronary arteries of the rats were ligated before 15 mg/kg nobiletin was injected intraperitoneally); (vi) nobiletin group of high dosage (AMI+NOB-H), n=10, (LAD coronary arteries of the rats were ligated before 30 mg/kg nobiletin was injected intraperitoneally); (vii) enalapril group (AMI+Enalapril), n=10, (LAD coronary arteries of the rats were ligated before 10 mg/kg enalapril was given intragastrically); echocardiograms were taken after the 21-day experiment.

1. Construction of a Myocardial Ischemia Rat Model

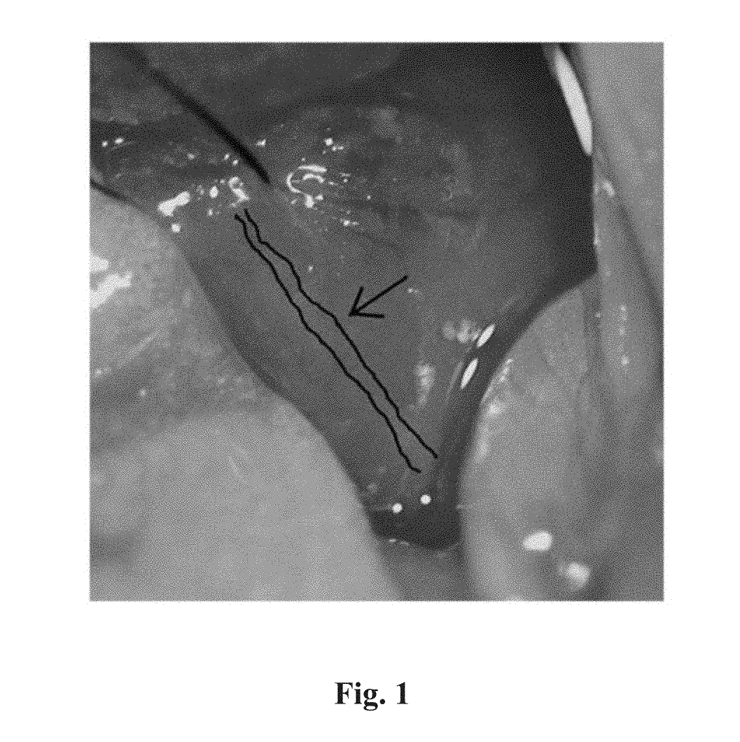

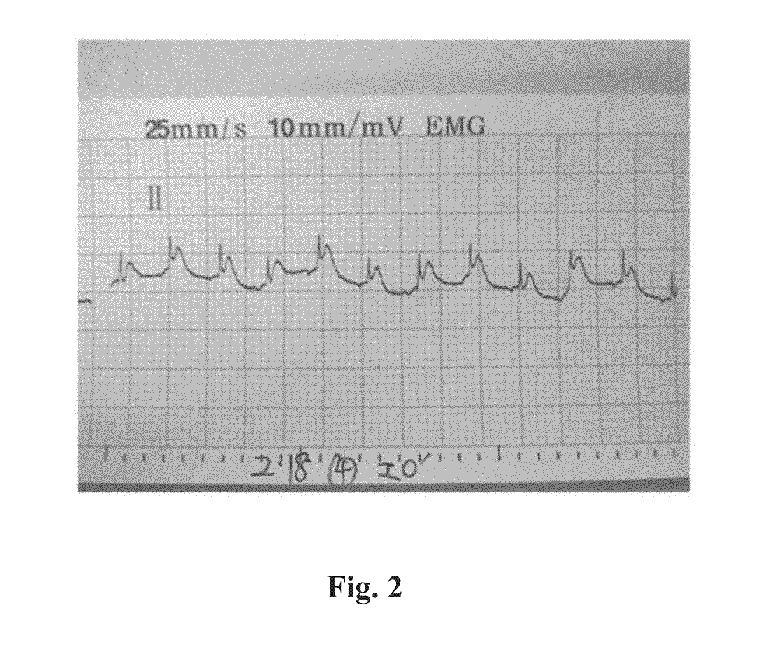

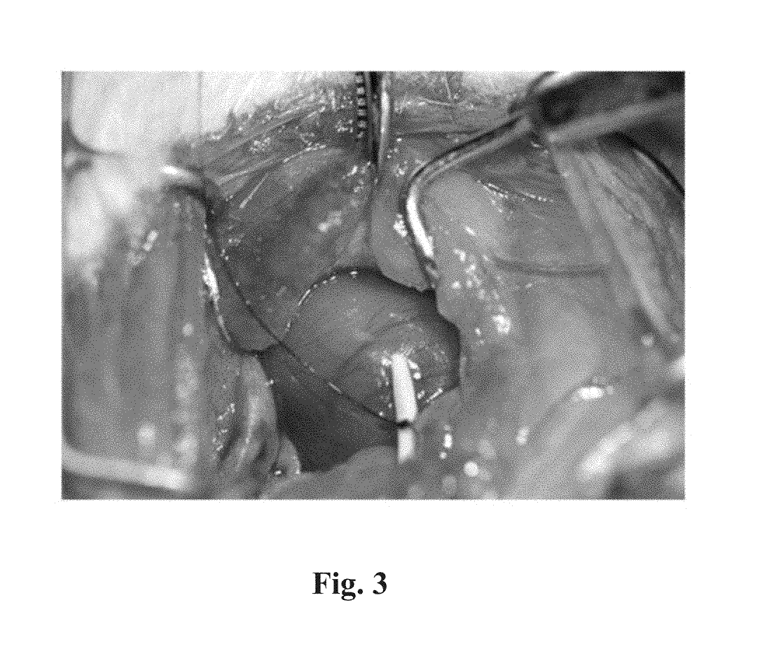

Male SD rats were weighed and then anaesthetized by intraperitoneal injections of 10% chloral hydrate (300 mg/kg). The rats were fixed on a table lying on their back, each connected with an electrocardiography and 2-lead electrocardiograms (ECGs) were recorded. The rats were inserted with trachea cannulas and artificially ventilated with a ventilator (positive airway pressure, frequency: 70 times/min, Ti/(I:E)=5:4, V_{T}: about 15 ml/kg). Sterilized the left parasternal skin on the third and forth intercostals regions, cut the skin and separated muscle layers trough blunt dissection, opened the thorax, then cut the pericardium to expose the left ventricle and the left auricle, the left ventricle coronary artery was seen descending from the middle of the left auricle to the anterior wall of the left ventricle (see FIG. 1). Using left coronary artery as a reference, between left auricle and pulmonary conus arteriosus, the left anterior descending (LAD) coronary arteries of the rats were ligated by NO. 6-0 sutures. ST segments rose in 2-lead electrocardiogram of the rat after LAD ligation (see FIG. 2), and myocardial ischemia symptoms such as the distal myocardial tissues turned pale after LAD ligation acted as an index of whether the ligation worked (see FIG. 3). Sham-treated rats underwent the same surgical procedure without the ligation of LAD coronary artery.

2: Echocardiography.

M-mode echocardiography was performed with a high-resolution imaging ultrasound system for small animals (Vevo 2100, Visual Sonics Inc., Canada; equipped with a RMV707B probe, operated in a frequency of 23 MHz or 30 MHz and penetrated to a depth of 10-15 mm) and connected to a Siemens ultrasonic instrument with a high-resolution probe 15L8 (Contrast Pulse Sequencing™, Sequoia 512, Siemens, Germany) at the day before the experiment ended. Rats were anaesthetized with 2% isoflurane gas and fixed on a heating plate maintained at 37° C. lying on their back, their limbs were connected to electrodes to monitor heat rates and record ECGs. Chemical sheering was performed on left chests of rats to reduce ultrasonic disturbance, then smeared ultrasonic coupling gel on the chests to better perform ultrasound test. M-mode echocardiography: the probe was placed on the chest wall corresponding to the breastbone to get views of parasternal long-axis view and parasternal short-axis. Under the view of left ventricle short axis, recorded the motion of the left ventricle at papillary muscle level in M-mode to measure left ventricular internal dimension, diastolic (LVIDD) and left ventricular internal dimension, systolic (LVIDS), calculate left ventricular end-systolic volumes (LVESV), left ventricular end-diastolic volumes (LVEDV), left ventricular ejection fraction (LVEF) and left ventricular fractional shortening (LVFS) using the maximum and minimal cross-sectional area and thickness. Relevant formulas are as follows.

LVEF (%)=(LVEDV−LVESV)/LVEDV×100%

LVFS (%)=[(LVIDD−LVIDS)/LVIDD]×100%

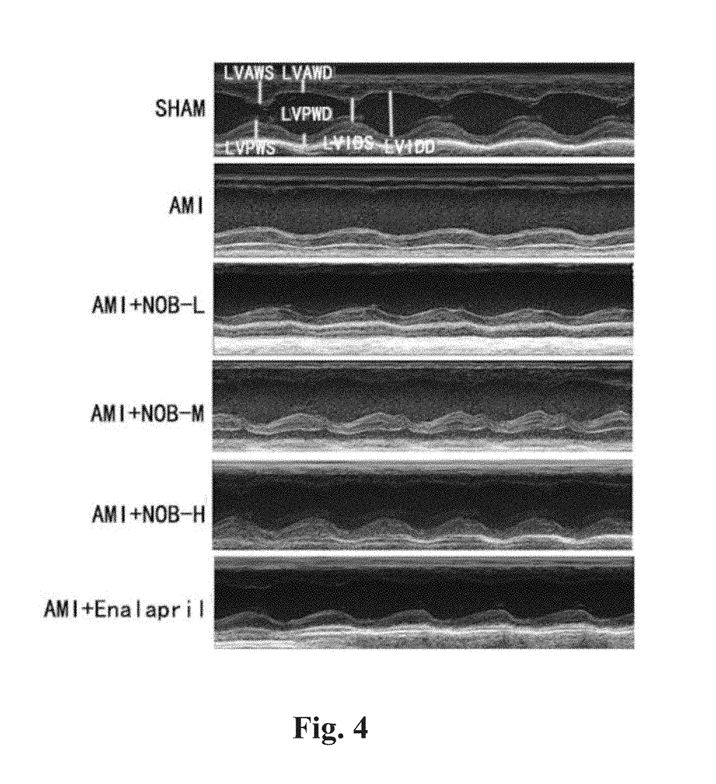

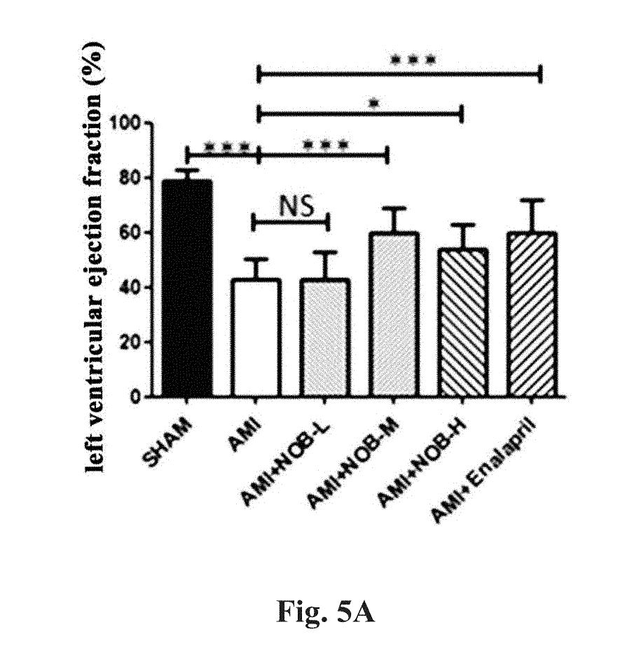

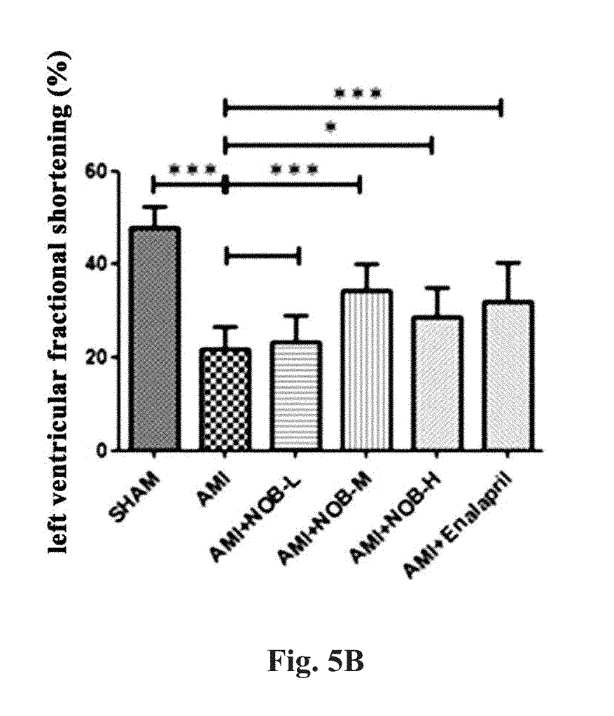

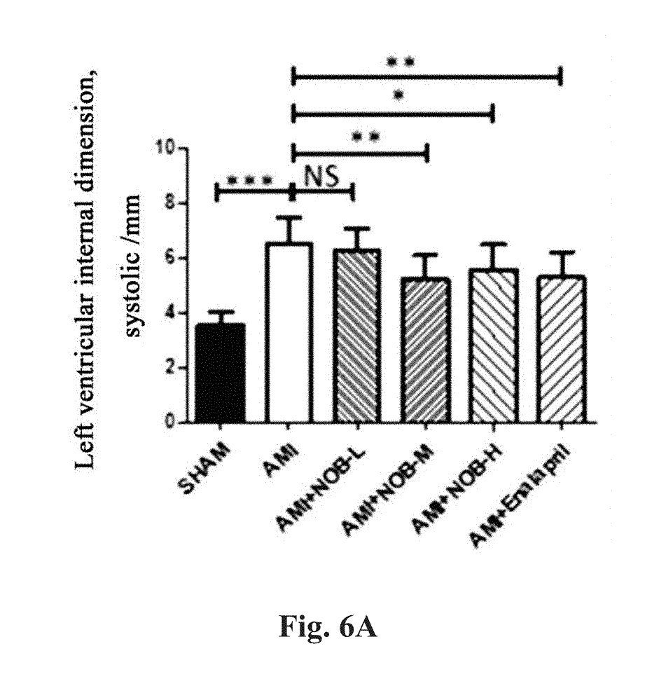

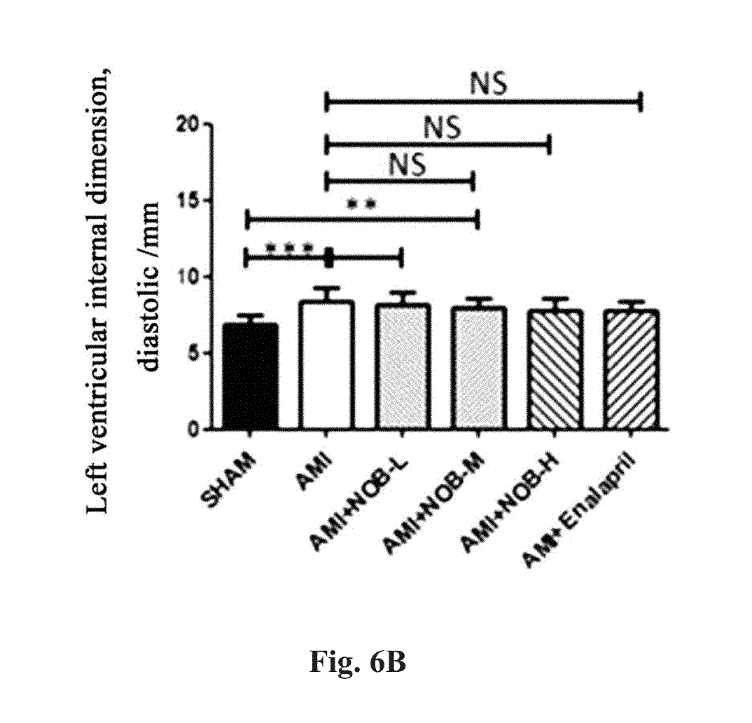

As shown in FIG. 4, 5A, 5B, 6A, 6B, compared to the sham group, the infarction group had significant decreased LVEF and LVFS, and significant increased LVIDD and LVIDS; compared to the infarction group, the medium dosage nobiletin group, high dosage nobiletion group and enalapril group significantly increased LVEF and LVFS, and significantly reduced LVIDS rises, but no significant reduction of LVIDD rises were seen; the results demonstrates that nobiletin mainly improves myocardial contractility.

Experiment 2. Determination of Infarct Size by TTC Staining.

Normal cardiomyocytes contain dehydrogenases which enable cardiomyocytes to deoxidize colorless triphenyl tetrazolium chloride (TTC), an oxidized dye, to red TTC in the presence of NADH, thus live myocardia are stained. When myocardial infarction occurs for some time, dehydrogenases in the cells were released and lost due to cell membrane damages. Because TTC is not deoxidized into a colored form, infarcted myocardium cannot be stained. Therefore unstained infracted myocardium and stained normal myocardium can be separated, and infarct size is estimated by weighing or quadrature of a slice area. Formula: Infarct size (%)=Σ (infarction area of the slice/myocardium area of the slice×myocardium weight of the slice)/left ventricle weight×100%.

The rats were sacrificed after heart functions were checked by echocardiography in Experiment 1. Hearts of the rats were separated and stained by TTC to determine the infarct sizes of the myocardia, specific procedures were as follows.

(1) Separating: The rats were anaesthetized with 1% sodium pentobarbital. Cut postcava and lavaged the hearts through arcus aortae. Quickly separated the hearts and then washed away residual blood using cold PBS.

(2) Triming: Removed atriums and right ventricles, restored left ventricles were weighed and recorded.

(3) Slicing: Quick-froze left ventricles in a refrigerator at −20□ for about 20 min, transversely sliced the ventricles into 4 slices from apex to basis of the hearts of 1-2 mm thick. Weighed and recorded each slices.

(4) Staining: Preheated the slices to 37° C. in 1% TTC hydrochloride buffer (pH=7.4, reused for twice) in brown bottles, then incubated the slices in dark in an incubator at 37° C. for 15 min. Turned over the bottles to allow myocardial tissues uniformly subjected to staining solution. After staining, infarct areas were white while non-infarct areas were deep red.

(5) Fixing: Sectioned Stained myocardium and fixed in 4% paraformaldehyde for 30 min.

(6) Imaging: Shot photos of myocardium arranged from the basis to the apexes of the hearts with a Canon digital camera.

(7) Analysis: Areas in different colors were measured by Image-Pro Plus, a image analysis software. Infarct level was expressed as a percentage of left ventricle weight (%=infarct area (shown as white area) weight/left ventricle weight).

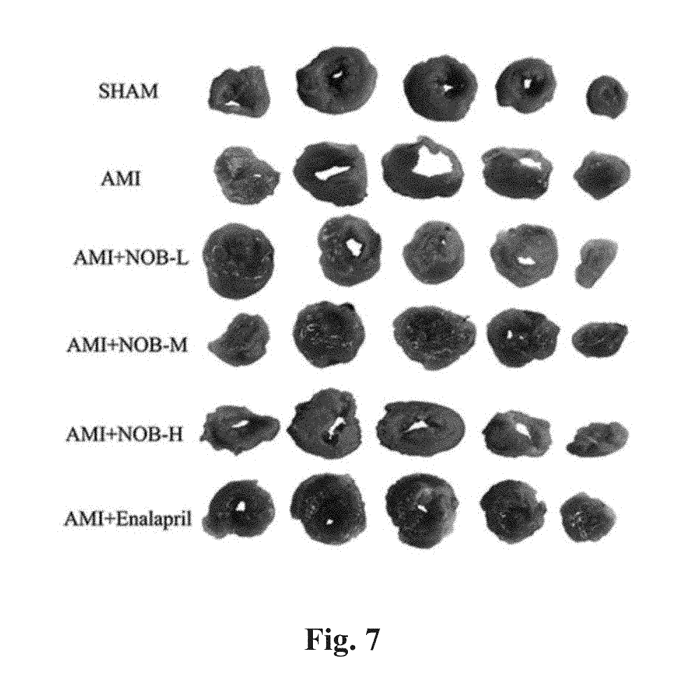

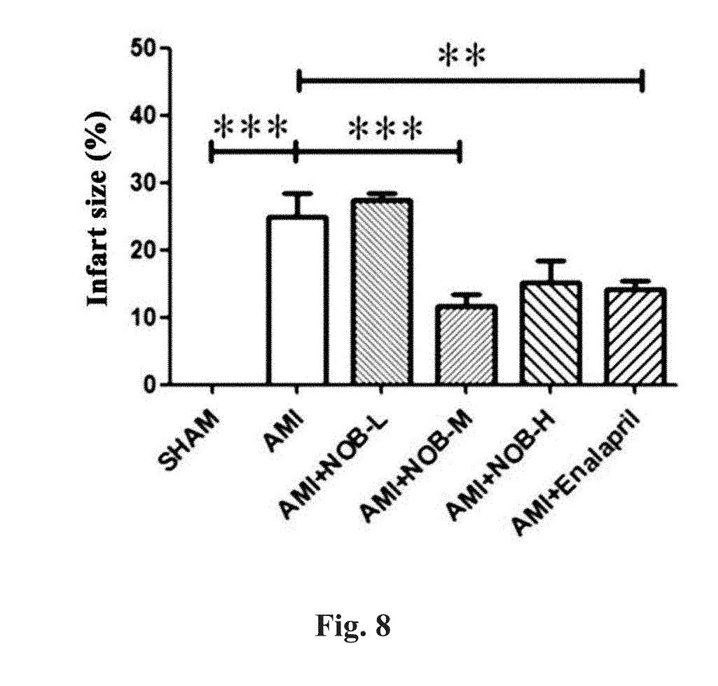

Original Figure of FIG. 7 is a color image in which clearly shows the white areas of myocardium due to myocardial infarction after staining, compared to the sham group, there were larger white infarct areas in the infarction group; white infarct areas in the nobiletin groups and enalapril group were smaller than that in the infarction group. FIG. 8 shows infarct area fractions of the rats, compared to the sham group, the infarction group had greater infarct area percentages; compared to the infarction group, the nobiletin groups and enalapril group had lower infarct area percentages.

Synthesizes the results of Experiment 1 and Experiment 2, enalapril, an antigiotensin converting enzyme inhibitor, is a drug for chronic heart failure; the nobiletin group of medium dosage (AMI+NOB-M) and the enalapril group (AMI+Enalapril) shared similar therapeutic effects against chronic heart failure.

Experiment 3. Effect of Nobiletin on Survival of Cardiomyocytes Subjected to Oxygen-Glucose Deprivation (OGD).

In vitro, H9C2 cells with OGD treatment were used to model myocardial ischemia. OGD treatment: the cells were cultured and subjected to OGD when the density of the cells reached to 80%-90%. Collected the OGD-treated cells from the medium and rinsed trice with glucose-free EBSS, after that cultured the cells in the same medium at 37° C. in an anoxia chamber saturated with 94% N2/5% CO₂/1% O₂ for a preset time.

Apoptosis Detection by Annex V/PI Staining.

After previous procedures, the cells were digested with 0.25% trypsin and collected to stain with both fluorescein isothiocyanate (FITC) labeled nectin (Annexin V-FITC) and PI (according to the manufacturer's protocol of Annexin V-FITC kits). Flow cytometry detection was carried out immediately under an excitation wavelength of 480 mm to detect 1×10⁴ cells. PI emits light of a wavelength about 623 nm (red fluorescence) while FITC emits light of about 520 nm (green fluorescence). Analyzed the detection data by CELLQEST Software and the results were shown in scatter plots (see FIG. 9A-9F) and statistics (see FIG. 10).

As shown in FIG. 9A-9F and FIG. 10, the results demonstrates that nobiletin significantly reduced apoptosis resulting from OGD in a dose-dependent manner.

It should be noticed that, aforementioned embodiments are only used to illustrate the technical scheme of the present invention, not to limit the scope of the present invention. Despite that the illustration is made in reference to preferable embodiments, those skilled in the art should understand that improvements, modifications and alterations are intended to be made and are intended to be within the spirit and scope of the present invention.

What is claimed is:

1. A method for preventing or treating heart failure, comprising
administrating nobiletin to a patient with heart failure.

2. The method for preventing or treating heart failure according to claim 1, wherein the nobiletin reverses cardiac remodeling after heart failure and ischemia.

3. The method for preventing or treating heart failure according to claim 2, wherein the reversion of the cardiac remodeling after heart failure and ischemia comprises reversing the increase of systolic ventricular internal dimension, reversing the decrease of ventricular ejection fraction and ventricular fractional shortening, reducing infarct size, and promoting cardiomyocyte survival rate.

4. The method for preventing or treating heart failure according to claim 1, wherein the heart failure is chronic heart failure.

5. A pharmaceutical composition for preventing or treating heart failure, comprising nobiletin and a pharmaceutically acceptable carrier.

6. The pharmaceutical composition according to claim 5, a dosage form of which is a liquid for injection or a lyophilized formulation.

7. A method for preventing or treating heart failure in a patient comprising administrating the pharmaceutical composition according to claim 5 to the patient.

8. A method for preventing or treating heart failure in a patient comprising administrating the pharmaceutical composition according to claim 6 to the patient.

9. The method for preventing or treating heart failure according to claim 7, wherein the pharmaceutical composition reverses cardiac remodeling after heart failure and ischemia.

10. The method for preventing or treating heart failure according to claim 8, wherein the pharmaceutical composition reverses cardiac remodeling after heart failure and ischemia.

11. The method for preventing or treating heart failure according to claim 9, wherein the reversion of the cardiac remodeling after heart failure and ischemia comprises reversing the increase of systolic ventricular internal dimension, reversing the decrease of ventricular ejection fraction and ventricular fractional shortening, reducing infarct size, and promoting cardiomyocyte survival rate.

12. The method for preventing or treating heart failure according to claim 10, wherein the reversion of the cardiac remodeling after heart failure and ischemia comprises reversing the increase of systolic ventricular internal diamention, reversing the decrease of ventricular ejection fraction and ventricular fractional shortening, reducing infarct size, and promoting cardiomyocyte survival rate.

13. The method for preventing or treating heart failure according to claim 7, wherein the heart failure is chronic heart failure.

14. The method for preventing or treating heart failure according to claim 8, wherein the heart failure is chronic heart failure.
